(19) United States
(12) Patent Application Publication
(10) Pub. No.: US 2012/0232381 A1
(43) Pub. Date: Sep. 13, 2012

(54) METHOD FOR NON-INVASIVE QUANTITATIVE ASSESSMENT OF RADIOACTIVE TRACER LEVELS IN THE BLOOD STREAM

(75) Inventors: Kenneth George Antonius Gilhuijs, Amsterdam (NL); Michiel Sinaasappel, Amsterdam (NL); Sara Hillegonda Muller, Amsterdam (NL)

(73) Assignee: STICHTING HET NEDERLANDS KANKER INSTITUUT, Amsterdam (NL)

(21) Appl. No.: 13/498,153

(22) PCT Filed: Sep. 24, 2010
(86) PCT No.: PCT/NL2010/050621

§ 371 Date: May 22, 2012

Related U.S. Application Data
(60) Provisional application No. 61/245,828, filed on Sep. 25, 2009.

(51) Int. Cl. A61B 6/00

(52) U.S. Cl. 600/425; 600/431

(57) ABSTRACT

A method and system for non-invasive quantitative assessment of radionuclide tracer levels in the blood stream. The method relies on the finding that the gamma-radiation signal acquired using a gamma scintillation probe, is the resultant from a wave-component with changing amplitude, resulting from the radiotracer in the bloodstream and a non-wave background component resulting from radiotracer distributed throughout the tissue surrounding the arterial vessel. The method involves phase-sensitive conversion of the ‘input signal’, extracting there from an output signal representing the signal component originating from the bloodstream, using the changes in arterial blood volume as the reference wave-form. A particular aspect of the invention concerns methods of quantitative PET or SPECT imaging wherein the concentration of radionuclides in the blood stream as a function of time after injection is assessed using the method of the invention.

### FIELD OF THE INVENTION

The present invention concerns a method for non-invasive quantitative assessment of radionuclide tracer levels in the blood stream, as well as the system for use in such methods. A particular aspect of the invention concerns methods of quantitative PET or SPECT imaging wherein the concentration of radionuclides in the blood stream as a function of time after injection is assessed using the method of the invention.

### BACKGROUND OF THE INVENTION

Positron emission tomography (PET) scanning and single photon emission computed tomography (SPECT) are diagnostic tools for non-invasively imaging living organisms, essential to the investigation of chemical and functional processes in biochemistry, biology, physiology, anatomy, molecular biology, and pharmacology. While techniques, such as x-rays, computed tomography (CT), and magnetic resonance imaging (MRI) provide anatomical images, PET and SPECT scanning techniques provide insight into biochemical changes that generally occur long before a corresponding structural change is detectable by more traditional techniques.

To use PET or SPECT quantitatively it is essential to quantify the processes of uptake and clearance of the radio tracer. The first step in the modelling of these processes is to accurately measure radiotracer concentrations in arterial blood as a function of time after injection, which is commonly referred to as an “arterial input function” (AIF) or “time course of activity concentration” (TCC).

Currently the most reliable method to assess the concentration of radionuclides in the blood stream involves repetitive withdrawal of arterial blood samples and subsequent measurement of the radionuclide concentration using a well cell counter. This method requires an invasive procedure extending up to an hour after injection of the tracer, e.g. by catheterization, to obtain discrete blood samples. Unfortunately, the invasive withdrawal of blood is a significant discomfort to the patient, as well as a significant health risk for both the patient and hospital personnel through exposure to blood borne diseases and radioactive contamination. Moreover, the blood samples need to be processed in a dedicated laboratory which is costly and relatively time-consuming. To circumvent the inconveniences and health risks associated with direct arterial blood sampling, several approaches have been examined in an effort to non-invasively obtain an accurate arterial input function. While some approaches have focused primarily on the use of tomography, others have examined additional detector systems that generate a quantitative image-derived input function.

One such approach is ‘dynamic scanning’ of the patient during and directly after the injection. This methodology requires the patients to be in the medical scanner for an extended period of time (up to an hour). This method is often logistically complicated and costly because the scanner cannot be employed for other patients during this time. Moreover, being in the scanner for extended amount of time can be a burden to the patient.

Other studies have examined the possibility of obtaining an input function using tomography and large blood vessel imaging. However, this approach is limited in several respects. First, tomography exhibits a partial volume effect defined by spatial resolution. Moreover, an artery large enough to provide reliable data may not be present in the field of view. Second, time resolution may be determined by frame acquisition rates specified for a particular study. Although list mode acquisition capabilities reduce restrictions associated with slower acquisition rates, many scanners do not have this capability available. Third, subject placement within the tomograph may affect the accuracy of the input function, and obtaining reproducible positioning of the body is difficult.

Another alternative is to use a standardized input function, which is averaged across many subjects, or a modelled input function. In the latter method, the input function is calculated from various physiological parameters. However, since the input function is very dependent on individual procedural variables and physiological states, such as differences in liver and kidney function, these methods may lead to inaccurate results.

It has also been suggested to use a portable positron or gamma counter for PET tracers, placed directly over a blood vessel or lung. Some of these methods are restricted to very specific applications. In the method proposed by Nelson et al (1993) for instance, the flux of photons emitted from the superior lobe of the right lung following an intravenous bolus of H₂¹⁵O is measured. This method is not applicable for measurement of the redistribution of radiotracers containing ¹⁸F.

Watabe et al (Watabe 1995) directly measured the emitted positrons (and not the photons produced after annihilation of the positron). Due to the short free path length of positrons, the technique can only be used when the detector is placed on top of the wrist artery. The measured signal will originate from the blood. The disadvantage of this technique is that the positrons need to have enough energy to reach the detector from the blood. This makes it suitable for ¹⁵O but not for ¹⁸F.

The device described in the Japanese patent “Device for measuring radioactivity in blood” (JP2594411) is in fact a mini PET scanner wrapped around the wrist, this modality is capable of measuring the arterial concentration of positron emitting probes. It is, however, a complicated and large setup and does not make use of the special temporal behavior of the arterial blood stream as described in the current study.

US 2005/0167599 discloses a PET wrist detector, which would be capable of non-invasively measuring the arterial concentration of positron emitting probes. It involves a rather complicated and large set-up.

The methods described in the prior art so-far, all have serious drawbacks, as will be clear from the foregoing, such that there is still a distinct need for an improved non-invasive methods for quantitative assessment of radioactive tracer levels in the blood stream. In particular, it still is a challenge to develop such a method that does not involve a large and complicated set-up while still being able to discriminate counts originating from the arterial vessels and the surrounding tissues. It is an objective of the present invention to realize this.

### SUMMARY OF THE INVENTION

The present inventors have found that quantitative non-invasive assessment of radioactive tracer levels in the blood stream can be realized with a method relying on phase-sensitive processing or detection of a gamma-counter signal obtained with a probe placed over e.g. an arterial vessel.

Such arterial vessels vary in diameter due to the pumping of the heart. When radiotracers are injected intravenously the radiotracers will distribute throughout the blood stream. A gamma counter placed over a part of the body containing arterial vessels will measure a time varying signal originating from arterial blood. The arterial blood concentration of radiotracer is assumed to be constant within one heartbeat, therefore causing the number of counts to vary with the changes in the volume of the arterial vessels. In addition, due to the redistribution of the radiotracer in other organs the concentration radiotracer in the blood will decrease and the concentration in the tissue surrounding the vessels will increase. The decrease in arterial blood level is reflected in a diminishing of the amplitude of the heartbeat-induced volume changes. Thus, theoretically, the gamma-radiation signal measured using a scintillation probe placed over e.g. an arterial vessel is the resultant of a wave-component with changing amplitude, resulting from the radiotracer in the bloodstream and a non-wave background component resulting from radiotracer distributed throughout the tissue surrounding the arterial vessel.

The present inventors have now established that it is feasible to quantitatively assess the radionuclide levels over time following administration, relying on these combined components making up the gamma-radiation ‘resultant signal’ acquired using a gamma scintillation probe. This is accomplished by phase-sensitive conversion of this ‘input signal’, extracting therefrom an output signal representing the signal component originating from the bloodstream, using the changes in arterial blood volume as the reference wave-form.

Because the method allows for quantitative monitoring of fast changes in tracer concentration, pharmacokinetics can be assessed while the uptake and clearance of tracer in the human system does not yet occur at equilibrium rates. This is a further major advantage over the currently existing methodologies, which require the pharmacokinetic system to be in equilibrium to assess pharmacokinetic rates of tracer transport. Especially when new antibody-labelled tracers are used, it will take a long time for the underlying pharmacokinetics to reach equilibrium state, thus rendering conventional pharmacokinetic modelling by dynamic scanning or blood withdrawal difficult to interpret. In the method provided by the present invention it would be feasible for patients to wear a small portable scanner for days if necessary.

The present system is also suitable for ¹⁸F radionuclide studies, which is more widely available and has much lower positron energy. As explained before, prior art non-invasive techniques are not suitable for use with ¹⁸F radionuclides.

Hence, the present invention provides methods of quantitative assessment of radionuclide levels in the bloodstream based on the above principle as well as the system that is suitable for use in these methods.

These and other aspects of the invention will be explained and illustrated in more detail in the below description and examples.

### DETAILED DESCRIPTION OF THE INVENTION

The first aspect of the invention concerns a method for quantitative assessment of radionuclide levels in the bloodstream of a subject following administration of said radionuclide to said subject, said method comprising:

acquiring an input radionuclide signal using a gamma scintillation counter with a probe placed over a portion of the subject's body;

feeding the input radionuclide signal to a processing unit;

phase sensitive processing, by said processing unit, of the input radionuclide signal, extracting from said input radionuclide signal an output signal representing the radionuclide concentration or activity in the bloodstream, wherein the phase sensitive processing uses the phase and/or frequency of arterial vessel volume changes as the reference waveform (parameters).

In this document and in its claims, the verb “to comprise” and its conjugations are used in their non-limiting sense to mean that items following the word are included, without excluding items not specifically mentioned. In addition, reference to an element by the indefinite article “a” or an does not exclude the possibility that more than one of the element is present, unless the context clearly requires that there be one and only one of the elements. The indefinite article “a” or an thus usually means “at least one”.

The method of the invention, in its broadest aspect relates to ‘quantitative assessment of radionuclide levels in the bloodstream’. More specifically, the radionuclide concentration or radionuclide activity values in the bloodstream at (one or more) given time points following administration are quantified. As will be understood by those skilled in the art, there is a direct (linear) relationship between the concentration and activity of the radionuclide at any given time point such that no distinction is made between these parameters in the context of the invention. The radionuclide concentration or activity values describe the availability of the radionuclide to one or more sites or regions of interest at the respective time points, which information is required for most, if not all, types of quantitative radiotracer studies.

In a preferred embodiment, a method as defined above is provided for obtaining a so-called time-activity or time-concentration curve or profile. Such a time-activity or time-concentration profile accurately represents the redistribution of the tracer from the bloodstream compartment to (an)other compartment(s). The time-activity or time-concentration profile may also sometimes be referred to as the ‘time course of activity concentration’ (TCC) or ‘arterial input function’ (AIF). An accurate profile or curve in accordance with the invention can be obtained by continuous or periodic acquisition of the radionuclide signal in accordance with the invention.

A particularly preferred aspect of the present invention concerns a method of quantitative assessment of radionuclide uptake by a tissue, e.g. tumorigenic tissue, brain tissue, vascular tissue and cardiac tissue, in a subject using PET or SPECT imaging performed after administration of said radionuclide to said subject, wherein the TCC or AIF is acquired using the method as defined herein. The present method may be used to provide the AIF or TCC in studying physiological functions in a healthy subject or in a subject suffering from a known or unknown disease or condition as well as in studying the behavior of (analogues) of endogenous or exogenous substances in healthy subjects or subjects suffering from a disease or condition. Such studies may be performed for scientific, therapeutic and/or diagnostic purposes, as will be understood by those skilled in the art. Examples of radionuclide studies are known to those skilled in the art, especially in the fields of oncology, neurology, cardiology, neuropsychology, psychiatry and pharmacology. Such examples include diagnosis, staging, and monitoring treatment of cancers, particularly in Hodgkin's disease, non Hodgkin's lymphoma, and lung cancer; differentiating Alzheimer's disease from other dementing processes, and early diagnosing of Alzheimer's disease; visualization of amyloid plaques in the brains of Alzheimer's patients; localization of seizure focus; visualization of neuroreceptor pools in the context of a plurality of neuropsychiatric and neurologic illnesses; atherosclerosis and vascular disease study; identifying so-called “hibernating myocardium” in clinical cardiology; imaging of atherosclerosis to detect patients at risk of stroke; examining links between specific psychological processes or disorders and brain activity; examining schizophrenia, substance abuse, mood disorders and other psychiatric conditions; studying biodistribution in pre-clinical trial of new drugs and studying drug occupancy at a purported site of action by competition studies. The present invention encompasses any such study wherein the present method is used to provide the AIF or TCC. The exact mathematic approaches for modeling the physiological functions of interest at one or more given sites in the body using the concentration or activity values acquired according to the present method are known to those skilled in the art, and the invention is not limited to any particular embodiment in this respect.

As will be understood the subject may be a human or an animal. Preferably said subject is a healthy human or a human suffering from a disease or condition, typically a human suffering from or at risk of cancer, alzeheimer's disease, cardiovascular diseases, neurological disorders and phychiatric disorders.

In a preferred aspect, the present invention provides a method of diagnosing a disease or condition, such as cancer, alzeheimer's disease, cardiovascular disease, neurological disorders and phychiatric disorders, using quantitative PET or SPECT scanning wherein the TCC or AIF is acquired using the method as defined herein.

In another preferred aspect, the present invention provides a method of monitoring treatment of a disease or condition, such as cancer, alzeheimer's disease, cardiovascular disease, neurological disorders and phychiatric disorders, using quantitative PET or SPECT scanning wherein the TCC or AIF is acquired using the method as defined herein.

A ‘radionuclide’, in the context of the present invention refers to an atom with an unstable nucleus, which is a nucleus characterized by excess energy which is available to be imparted either to a newly-created radiation particle within the nucleus, or else to an atomic electron (see internal conversion). The radionuclide undergoes radioactive decay, thereby emits a gamma ray(s) and/or subatomic particles. These particles constitute ionizing radiation. Radionuclides are often referred to in the art as radioactive isotopes or radioisotopes and, for the purposes of the present invention, these terms are deemed interchangeable. Radionuclides used in PET or SPECT scanning are typically isotopes which upon decay emit gamma rays (or, in the case of PET, positrons) with relatively short half lives such as carbon-11 (¹¹C), which has a half life of approximately 20 min., nitrogen (¹³N), which has a half life of approximately 10 min., oxygen-15 (¹⁵O), which has a half life of approximately 2 min., and fluorine-18 (¹⁸F), which has a half life of approximately 110 min. In principle any type of radionuclides may be employed in accordance with the present invention. However, as noted before, a particular advantage of the present invention vis á vis certain prior art methods resided in the possibility of employing ¹⁸F radionuclides. Hence, a preferred embodiment of the invention concerns a method as defined herein before wherein the radionuclide is a ¹⁸F radionuclide.

These radionuclides are typically incorporated into substances that somehow interact with a physiological process in the subject, such that the occurrence of said process and/or the rate thereof can be ‘visualised’. Such substances including a radionuclide may be referred to in the art as ‘radioactive tracer’, ‘radionuclide tracer’, ‘tracer’ or the like. These terms are deemed entirely interchangeable in the context of this specification. In accordance with the present invention the radionuclide tracer may in principle be any type of substance somehow interacting with the physiological processes taking place in a healthy human or animal or any process occurring as a result of a disease or condition in such a human or animal. Preferred examples of radionuclides in accordance with the present invention include glucose or analogues of glucose, water, ammonia or any kind of (drug) substance. Examples of such (drug) substances include chemotherapeutics, receptor ligands, enzyme substrates, enzymes, antibodies, antigens, etc., as well as analogues of the afore-mentioned substances. As will be understood by those skilled in the art, the present method is also particularly suitable for use in pharmacodynamic and pharmacokinetic studies of novel drug substances. Hence the invention is not limited to any exemplary drug substance currently known or available.

A typical radionuclide tracer study involves administration of the tracer into the body by intravenous injection in liquid or aggregate form, ingestion while combined with food, inhalation as a gas or aerosol, or rarely, injection of a radionuclide that has undergone micro-encapsulation. The mode and route of administration of the radioactive tracer is not particularly critical in accordance with the invention, although it should be borne in mind that the selected route and mode of administration should allow for a sufficient amount of activity to reach the site of interest taking into account the half-life of the selected radio-nuclide and the pharmakokinetic properties of the tracer. Intravenous administration will be the most convenient route because, in general, it will constitute the quickest method of delivering the tracer to the site of interest and involves the least complex absorption and distribution kinetics, although the suitability of the present method is not restricted to any specific pharmacokinetic model or phase, as noted before. A preferred embodiment of the invention provides a method as defined herein before wherein the radionuclide is administered through intravenous injection.

In accordance with the present invention acquiring the radionuclide signal involves the use of a gamma scintillation counter, typically comprising a sensor or probe, containing a transparent crystal that fluoresces when struck by ionizing gamma radiation, and a photomultiplier tube measuring the light from the crystal. The photomultiplier tube may be attached to an electronic amplifier and other electronic equipment. In accordance with the invention, the scintillation probe is placed over a portion of the subject's body during data acquisition, e.g. when the subject is placed in the PET or SPECT apparatus.

An essential aspect of the present invention is the phase sensitive processing (or demodulation) of the input radionuclide signal acquired with the scintillation probe, wherein an output signal representing the radionuclide signal originating from the bloodstream is extracted from said input signal. Put differently, the input radionuclide signal acquired using the gamma scintillation counter is separated into the background radionuclide signal representing radionuclide activity in the tissues surrounding the arterial vessel(s) at the site of signal acquisition and the output signal representing the radionuclide activity in the arterial bloodstream. These signals can be distinguished from each other because they differ in wave-form or, moreover, in the fact that the radionuclide activity in the bloodstream produces a wave-form (AC) signal, following the frequency (and amplitude) of arterial volume changes, constituting the reference wave-form, whereas the radionuclide activity in the surrounding tissue produces a non-wave-form (DC) signal. Phase sensitive processing relies on the orthogonality of wave-form functions. Specifically, when a wave-form function of frequency v is multiplied by another wave-form function of frequency μ not equal to v and integrated over a time much longer than the period of the two functions, the result is zero. In the case when μ is equal to v, and the two functions are in phase, the average value is equal to half of the product of the amplitudes. In essence, the phase sensitive processing takes the input signal, multiplies it by the reference signal, and integrates it over a specified time, usually on the order of milliseconds to a few seconds. The resulting output signal is an essentially DC signal, where the contribution from any signal that is not at the same frequency as the reference signal is attenuated essentially to zero, as well as the out-of-phase component of the signal that has the same frequency as the reference signal (because sine functions are orthogonal to the cosine functions of the same frequency).

This principle is also sometimes referred to in the art as lock-in detection'. In an embodiment of the invention an analogue lock-in detector is used for demodulating the input radionuclide signal in accordance with the foregoing. Typically, a lock-in detector or amplifier, or ‘phase-sensitive detector’, is essentially a homodyne with an electronic circuit functioning as a low pass filter, which is controlled by the reference waveform that caused the signal to be modulated, in casu the pulsatile arterial volume changes. The lock-in detector thereby effectively responds to signals which are coherent (same frequency and phase) with the reference waveform and rejects all others. Suitable lock-in detectors are available commercially. In another, equally preferred, embodiment of the invention, digital phase-sensitive processing is envisaged, using an algorithm functioning as the low-pass filter, in a manner essentially corresponding to the analogue low-pass filter. Computer programs suitable for this purpose are available commercially, for example LabView and MatLab. Hence in one embodiment of the invention the processing unit is an analogue circuit dedicated to perform the phase-sensitive processing of the input radio-nuclide signal. In another, equally preferred embodiment, the processing unit is a processor coupled to a memory, said processor being programmed to perform the phase-sensitive processing of the input radionuclide signal.

In accordance with the invention, frequency and phase of the reference wave-form, i.e. the arterial blood volume pulsations resulting from the beating of the heart, can be derived from the input radionuclide signal itself or they can be determined by simultaneous, typically non-invasive, measurement of a secondary parameter that is directly related to said arterial volume changes. A particularly suitable example of such a secondary parameter is near-infrared (NIR) absorbance or reflection, relying on the NIR light scattering and absorption by red blood cells in the arterial vessels, especially those vessels located near the surface of the subject's body. Thus, one embodiment of the invention provides a method as defined herein before, wherein the reference wave-form is derived from the signal acquired with the gamma scintillation counter and another embodiment of the invention provides a method as defined herein before wherein the reference wave-form is derived from simultaneous near-infrared (NIR) reflectance or absorption measurement using a ‘NIR probe’ placed over a portion of the subject's body.

For this purpose, a pulse oximeter is a particularly suitable device, which is commercially available as such. A pulse-oximeter, typically comprises a pair of small light-emitting diodes (LEDs) facing a photodiode through a translucent part of the patient's body, usually a fingertip or an earlobe. Typically, one LED is red, with wavelength of, for example, 660 nm, and the other is infrared having a wave-length of e.g. 905, 910, or 940 nm. The monitored signal from the photodiode pulsates with the heart beat because the arterial blood vessels expand and contract with each heartbeat, resulting in a periodic variation of infrared absorption over time. Hence, frequency and phase of the arterial blood volume pulsation can be detected. As will be understood by those skilled in the art, the present method, as it does not require any information on oxy/deoxyhemoglobin ratio's, allows for the use of variants of such pulse-oximeters, e.g. comprising a single NIR light source and/or different types of NIR light sources. Furthermore, in an alternative embodiment of the invention, an arrangement is used wherein the LED(s) and the photodiode are placed on the same surface, relying on reflectance rather than transmission of non-absorbed near infrared light. In a preferred embodiment of the invention the NIR probe is places over a portion of the subject's body where arterial vessels run near the skin surface. Most preferably a portion is selected which allows for NIR transmission measurement, such as the finger tip.

Further alternatives for simultaneously determining the frequency and phase of the pulsatile arterial blood volume, are also encompassed by the present invention. As will be understood by those skilled in the art though, it is preferred, with a view to patient logistics, that a device or method is used that can be applied within the confined space of e.g. a PET or SPECT apparatus and/or that can be applied for substantial periods of time, e.g. up to days if necessary. A second aspect of the present invention concerns a system for quantitatively assessing the radionuclide concentration in the bloodstream of a subject, said system comprising

a gamma scintillation counter with a probe adapted to be placed over a portion of the subject's body;

a processing unit interfaced with the gamma scintillation counter, wherein the processing unit is programmed or dedicated to receive an input radionuclide signals from said gamma scintillation counter and to perform the task of phase sensitive processing of the input radionuclide signal, extracting from said signal an output signal representing the radionuclide concentration or activity in the bloodstream, wherein the phase sensitive processing uses the phase and frequency of arterial vessel volume changes as the reference waveform.

As used herein the term ‘system’ refers to a collection of two or more hardware components, typically gamma scintillation counter hardware components and a processing unit and optionally further hardware components, that are interfaced and/or adapted in such a way that they can perform the task of phase sensitive processing of the gamma counter signal, as explained in the foregoing. Preferably the term refers to such a collection of hardware components which are integrated and combined in a housing such as to form a single piece of equipment. Other embodiments wherein several pieces of equipment, e.g. a gamma scintillation counter, a lock-in detector and/or a computer, optionally in combination with a pulse-oximeter, are combined, are also within the scope of the invention.

As noted before, the gamma scintillation counter comprises a sensor or probe, containing a transparent crystal that fluoresces when struck by ionizing gamma radiation, and a photomultiplier tube measuring the light emitted from the crystal and, typically, an electronic amplifier. The probe is typically adapted to be placed over a portion of the subject's body, preferably a portion comprising arterial vessels near the skin surface and/or a portion which is easily accessible for the probe, even in the confined space of a PET or SPECT apparatus, such as the upper arm, the elbow, the lower arm, the wrist, the hand, fingers, the neck, or the limbs. Most preferably the probe is adapted to be placed over and/or to be mounted on the subject's finger, especially the finger tip, or wrist. Furthermore, it is preferred that the system comprises the probe and the photomultiplier and, optionally, the amplifier and further electronic components in a single housing, preferably a portable housing which further comprises one or more aids for mounting it on the subject's body, such as a belt, a strap, a clip or a fastener.

As already explained in the foregoing the present invention resides in the finding that the radionuclide activity count can be processed phase-sensitively with the arterial pulsation as the reference wave-form, which can be achieved using an analogue or a digital low-pass filter', i.e. to respond to signals which are coherent (same frequency and phase) with the reference waveform while rejecting all others. Hence in one embodiment of the invention, a system is provided wherein said processing unit is an analogues circuit dedicated to perform said task of phase-sensitive processing of the radionuclide data. In another equally preferred embodiment a system is provided wherein said processing unit is a processor programmed to perform said task.

Typically, the present system comprises a computer arrangement for operating it, said computer arrangement typically comprising a processing unit connected to one or more memory units, which store instructions and data, and, optionally one or more reading units (to read, e.g., floppy disks, CD ROM's, DVD's, memory cards), input devices such as a keyboard, a mouse, a trackball, a touch screen or a scanner and/or output devices such as a monitor or a display. Further, a network I/O device may be provided for a connection to a network. The memory units may comprise RAM, (E)EPROM, ROM, tape unit, and hard disk. However, it should be understood that there may be provided more and/or other memory units known to persons skilled in the art. Moreover, one or more of them may be physically located remote from the processor, if required. The system may comprise several processor units functioning in parallel or controlled by one main processor, that may be located remotely from one another, as is known to persons skilled in the art. As explained before, the system comprises functionality either in hardware or software components to carry out its specific task. Skilled persons will appreciate that the functionality of the present invention may also be accomplished by a combination of hardware and software components. As known by persons skilled in the art, hardware components, either analogous or digital, may be present within the host processor or may be present as separate circuits which are interfaced with the host processor. Further it will be appreciated by persons skilled in the art that software components may be present in a memory region of the host processor. Typically a computer arrangement is included in the system, capable of executing a computer program (or program code) residing on a computer-readable medium which after being loaded in the computer arrangement allows the computer arrangement to carry out the method of the present invention.

In a preferred embodiment of the invention a system as defined herein before is provided further comprising a NIR absorption or reflectance measurement apparatus, with a probe adapted to be placed over a portion of the subject's body. As explained before a NIR absorption or reflectance measurement apparatus typically comprises a near infrared light source, such as a near infrared LED, and a photodiode, which are positioned relative to each other in such a way that the photodiode detects the light transmitted through said portion of the subject's body or reflected there from. Preferably the system of the invention is adapted, e.g. programmed, to process the NIR absorption or reflectance data in accordance with the method of the invention and/or to store the NIR absorption or reflectance data on a memory.

In accordance with a preferred embodiment of the invention a system is provided comprising the processing unit and the gamma scintillation counter components in a single apparatus. In an embodiment of the invention a system is provided as defined herein before comprising all its hardware components in a single housing, preferably a portable housing that can be worn by a subject, which housing further comprises one or more aids for mounting it on the subject's body, such as a belt, a strap, a clip or a fastener. Alternative embodiments are envisaged wherein the components to be placed in contact with the surface of the subject's body and the processing unit are incorporated in or as separate parts interconnected by a wire or adapted to communicate through a wireless connection. Preferably at least one surface of the part containing the component(s) to be placed in contact with the surface of the subject's body is shaped such as to be complementary to the portion of the body on which the housing is to be mounted and/or contains one or more aids for mounting it on the body. As will be understood by those skilled in the art, the system also typically comprises a power supply, e.g. in the form of a battery, and/or means for connecting it to an external power supply.

Another aspect of the invention concerns a computer program on a computer-readable medium to be loaded by a computer system comprising a memory and a processor, the processor being coupled to the memory, wherein the computer program product after being loaded allows the processor to carry out the task of computing the radionuclide levels in the bloodstream of a subject over time on the basis of an input radionuclide signal obtained from said subject after injection of radionuclide to said subject, by phase sensitive processing of the input radionuclide signal, extracting there from an output signal representing the radionuclide concentration or activity in the bloodstream, wherein the phase sensitive conversion/processing uses the frequency/phase of arterial vessel volume changes as the reference waveform. In a preferred embodiment of the invention, the computer program allows the processor to carry out said task using NIR reflectance or absorption data to determine the reference wave-form in accordance with the foregoing.

Also, in a preferred embodiment of the invention a computer program is provided that allows for a computer (processor) to carry out the task to model one or more physiological functions on the basis of PET or SPECT images using the radionuclide concentration- or activity-time profile obtained in accordance with the invention as the TCC or AIF. In a preferred embodiment this task is performed by a computer or workstation which is or can be interfaced with the PET or SPECT apparatus and the system of the present invention, such as to receive the PET or SPECT data as well as the TCC or AIF data.

For example, the invention may take the form of a computer program containing one or more sequences of machine-readable instructions describing a method as disclosed above, or a data storage medium (e.g. semiconductor memory, magnetic or optical disk) having such a computer program stored therein.

### DESCRIPTION OF THE FIGURES

FIG. 1: Graph showing simultaneously recorded radiotracer signal (with the gamma counter and NIR) on the fingers of a patient using a prototype of the device that implements the methodology described herein. The dots (A) indicate the raw counter signal (radiotracer). The thick interrupted line (B) and the thin interrupted line (C) show the smoothed counter signal and the NIR signal respectively. The data were obtained from a patient injected with ⁹⁹Tc bound to Human Serum Albumin. The signals each describe a single cycle of a heartbeat, and the smoothed gamma counter data closely resemble the NIR curve.

FIG. 2: Graph showing simultaneously recorded radiotracer signal (with the gamma counter and NIR) on the fingers of a patient using a prototype of the device that implements the methodology described herein. The dots indicate the smoothed and averaged counter signal (FSD), the squares represent the smoothed and averaged near infrared signal (NIR) signal. The data were obtained from a patient injected with ⁹⁹Tc bound to Human Serum Albumin. To obtain the above graph 10 portions of 3.5 seconds from the raw signals were averaged.

FIG. 3: Graph showing redistribution of the radiotracer in the bloodstream measured by the prototype device (2), the redistribution measured by concurrent dynamic scanning in a PET scanner (3), and the non processed signal from the gamma counter (1). The signal derived from the prototype device closely resembles the signal measured in the PET scanner.

FIG. 4: Graph showing redistribution of the radiotracer in the bloodstream measured by the prototype device. FIGS. 4A and 4C show the phase sensitive signal acquired in the knee as compared to the measurement acquired with the PET from the carotic artery. FIGS. 4B and 4D show the low pass filtered signal from the probe compared to the PET signal from the musculature in the neck.

### EXPERIMENTAL

### Experiment 1

Arterial vessels vary in diameter due to the pumping movement of the heart. When radiotracers are injected intravenously the radiotracers will dissolve in the blood stream. A gamma counter placed over a part of the body containing arterial vessels will measure a time varying volume of arterial blood. The concentration of radiotracer is assumed to be constant within one heartbeat, therefore causing the number of counts to vary with the changes in the volume of the arterial vessels. In addition, due to the redistribution of the radiotracer in other organs the concentration radiotracer in the blood will decrease. This decrease is reflected in a diminishing of the amplitude of the heartbeat-induced volume changes.

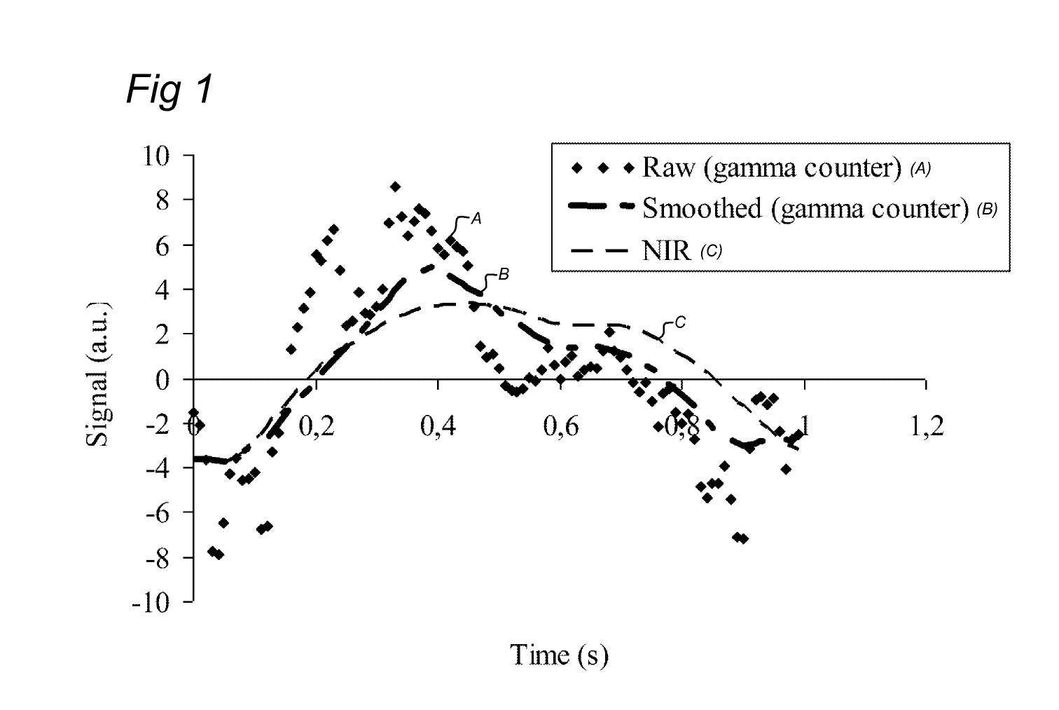

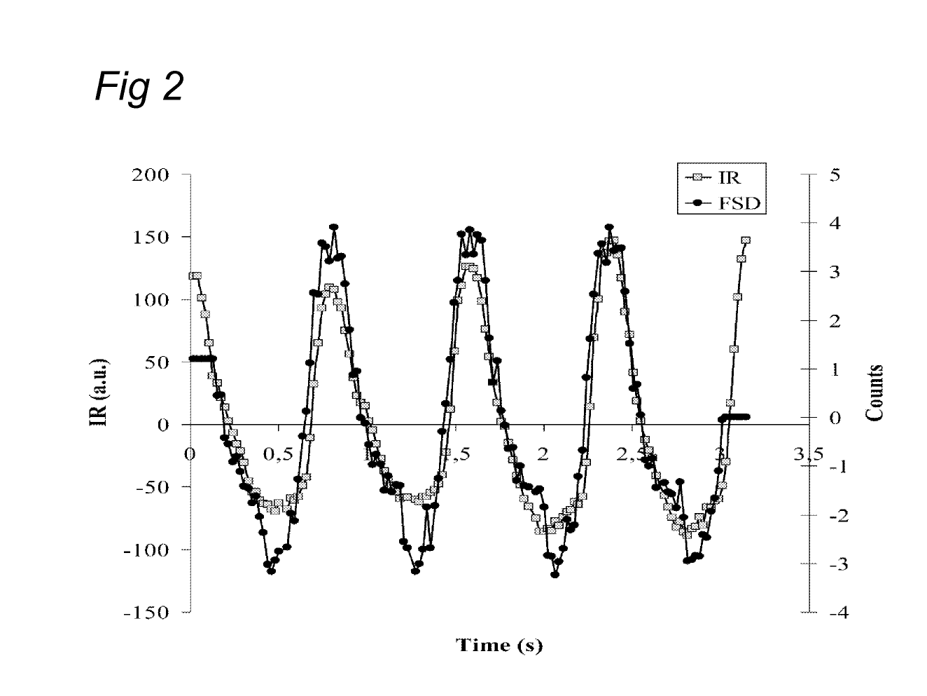

A change in the amount of circulating radioactive tracer is measured using phase-sensitive detection of the time-varying signal and the changing arterial blood volume. To validate that the time-varying signal is indeed caused by volume changes of the arterial blood vessels also the volume changes (in the finger) with near-infrared reflectance (NIR) measurement were measured. In the latter technique, which is also used in pulse-oxymeters, the light scattering and absorption of the red blood cells is used to measure the volume changes of the arterial blood caused by the beating heat (FIG. 1 and FIG. 2).

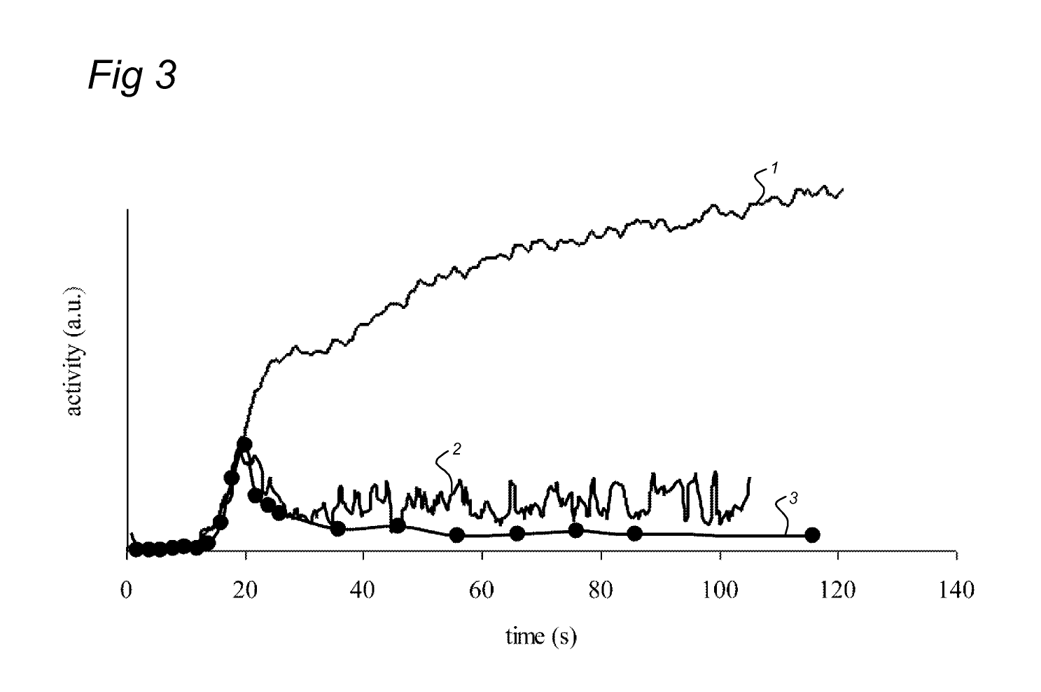

Using the radiotracer signal and the NIR in a phase sensitive detector yields a signal representing the modulation depth of the time varying radiotracer signal. This modulation depth is a measure for the difference in concentration over the vessel wall. FIG. 3 shows the result using the above method during and following the injection of radiotracer in a patient.

The radiotracer signal is measured using a standard gamma counter consisting of a NaI crystal on top of a photo-multiplier tube (PMT) with a preamplifier. The signal is fed into a counter, and the counts are stored in a PC. The applied counter also contains a 14-bit digitizer. This digitizer is used to measure the NIR signal. Because both signals are recorded with the same digitizer, the signals can be measured simultaneously.

The NIR signal is used as a reference signal to analyze the radiotracer signal. The amplitude of the time varying part of the radiotracer signal is then recorded as function of time (FIG. 3). FIG. 3 clearly illustrates a relation between the time varying radiotracer signal and the redistribution of the radiotracer from the blood to the surrounding tissues. The graph shows the increase in total counts measured (where the counter was placed on the leg near the knee). The initial fast rise is due to mixing of arterial and venous blood (where the injection itself takes place in much less than 5 sec). At the 60 second mark the mixing is completed and the signal becomes dominated by uptake in the musculature. The graph shows the time-dependent signal extracted from the device using the methodology described above. Here we can see a first rapid increase associated with the injected bolus. Then the radiotracer starts to leave the vasculature and equilibrium between the intra and extra vascular compartment becomes apparent. The signal disappears because the time varying signal is only above zero when there is a concentration difference over the vessel wall. Apparently this concentration difference has disappeared after the 60 seconds point.

The results obtained from the device were verified by comparing them with those from dynamic PET scanning (i.e., one of the methods described in the introductory part here before, which is used in this example as the reference method) obtained concurrently in the time period between tracer injection and 20 minutes thereafter. FIG. 3 shows that the redistribution of radiotracer from the blood stream obtained using the device is in accurate agreement with the redistribution derived from dynamic PET scanning. These results thus suggest that the device may be used as an off-line alternative to dynamic scanning at PET to measure redistribution of radiotracer from the blood stream.

### Experiment 2

Fluorine-18 labelled fluoroazomycin arabinoside ([¹⁸H]FAZA), has been developed as an PET tracer for assessment of Tumour hypoxia. For evaluation of the distribution over time of this tracer dynamic Pet scans have been performed during the first 20 minutes after administration of the tracer. Patients were measured twice in two consecutive days. To evaluate the Phase sensitive detector, we placed the probe between de knees of the patient while the dynamic PET scan was performed. The dynamic scan was constructed in time frames with a duration of 2 seconds. One ROI was placed over the left carotic artery an other ROI over an adjacent muscle.

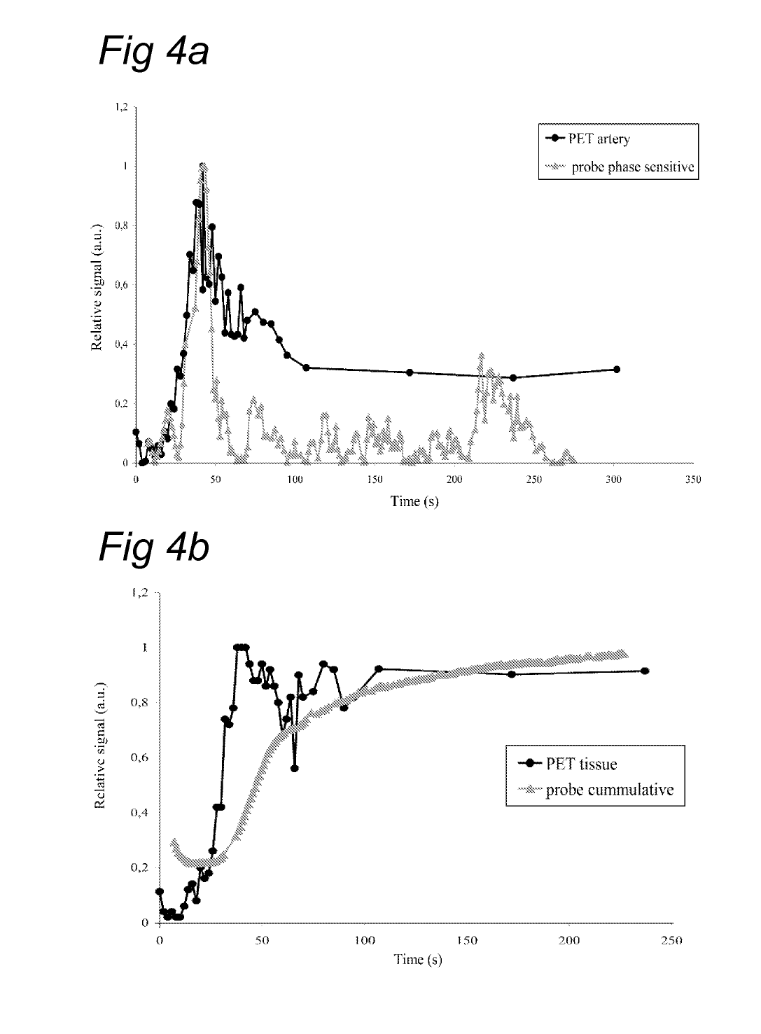

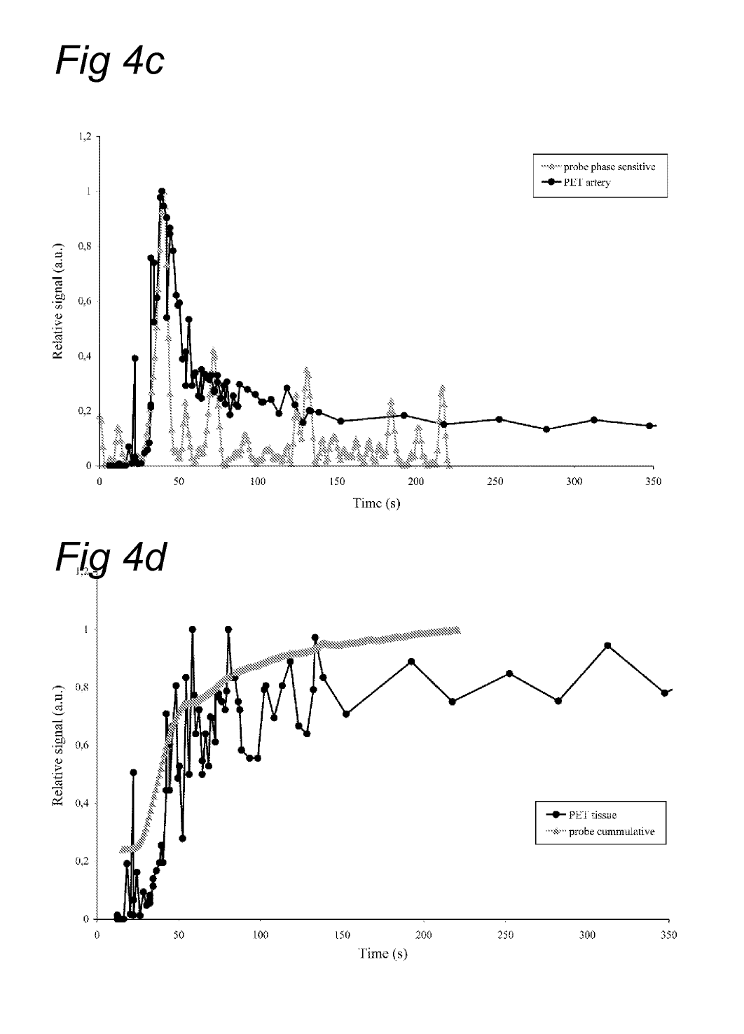

FIGS. 4A and 4C show the phase sensitive signal acquired in the knee as compared to the measurement acquired with the PET from the carotic artery. FIGS. 4B and 4D show the low pass filtered signal from the probe compared to the PET signal from the musculature in the neck.

### CONCLUSIONS

A technique has successfully been developed to non-invasively measure the concentration of radionuclides in the blood stream as function of time after injection without dynamic imaging. In the above experiments 1 and 2 it is demonstrated that this approach is feasible and fast. The described technique will be of great value when implemented in a portable device that complements medical PET and SPECT scanners. Redistribution rates are essential for the pharmacokinetic modelling used to improve the assessment of radiotracer uptake in tumours.

The device can be used as an accessory that does not require any changes to the current patient logistics. The data collected by the device could be readout by a nuclear medicine workstation and used to automatically correct the imaging values for, e.g., tumor glucose consumption and cell metabolism. As a result, tumor function will be evaluated with higher precision and less variation, thus requiring fewer patients to demonstrate potential benefit of one tracer over another. Manufacturers of new radionuclide tracers that target specific biomarkers can benefit from this approach because it will require fewer patients to perform phase-III trials, thus reducing the costs of tracer development. Moreover, achieving higher precision to evaluate tumor response to therapy may allow earlier switching from ineffective therapy to a more effective regimen, expediting valorisation of patient-tailored drug development.

Because the device is capable of quantifying fast changes in tracer concentration, it is possible to measure pharmacokinetics while the uptake and clearance of tracer in the human system does not yet occur at equilibrium rates. We believe that this is a major advantage, because current methodologies to assess pharmacokinetic rates of tracer transport require the pharmacokinetic system to be in equilibrium. Especially when new antibody-labelled tracers are used, it will take a long time for the underlying pharmacokinetics to reach equilibrium state, thus rendering conventional pharmacokinetic modelling by dynamic scanning or blood withdrawal difficult to interpret. Using the reported approach, patients could wear a small portable scanner for days if necessary.

In conclusion, it is feasible to quantify the concentration of radionuclides in the blood stream as function of time after injection by analyzing the pulsation of arterial vessels, combining NIR measurements with gamma-probe measurements. The method is expected to result in applications that lead to more efficient use of medical radionuclide scanners while improving the sensitivity and precision of these resources (short term). Moreover, implementation is expected to facilitate in expediting translational research of new molecular tracers (e.g., antibody-labelled tracers) while reducing its cost (long term). Because the reported technique is simple and relatively straightforward to build, it can be applied at large scale.

### REFERENCES

Noninvasive arterial monitor for quantitave oxygen/15/water bloodflow studies. A. Dennis Nelson et al. The Journal of Nuclear Medicine vol 34 no. 6 June 1993
Development of skin surface radiation detector system to monitor radioactivity is arterial blood along with positron emission tomography. Hiroshi Watabe et al. IEEE transactions on nucleaur science vol 42, no 4, August 1995.

1. System for quantitatively assessing the radionuclide levels in the bloodstream of a subject, said system comprising
a gamma scintillation counter with a probe adapted to be placed over a portion of the subject's body;
a processing unit interfaced with the gamma scintillation counter, wherein the processing unit is programmed or dedicated to receive an input radionuclide signal from said gamma scintillation counter and to perform the task of phase sensitive processing of the input radionuclide signal, extracting from said input radionuclide signal an output signal representing the radionuclide concentration in the bloodstream, wherein the phase sensitive processing uses the phase and frequency of arterial vessel volume changes as the reference waveform.

2. System according to claim 1, further comprising a NIR measurement apparatus, with a probe adapted to be placed over a portion of the subject's body.

3. System according to claim 1, wherein the gamma scintillation counter probe is adapted to be placed over a subject's finger.

4. Computer program on a computer-readable medium to be loaded by a computer system comprising a memory and a processor, the processor being coupled to the memory, wherein the computer program product after being loaded allows the processor to carry out the task of computing the radionuclide levels in the bloodstream of a subject over time on the basis of an input radionuclide signal obtained from said subject after injection of radionuclide to said subject, by phase sensitive processing of the input radionuclide signal, extracting there from an output signal representing the radionuclide concentration in the bloodstream, wherein the phase sensitive conversion/processing uses the frequency/phase of arterial vessel volume changes as the reference waveform.

5. Computer-readable medium being provided with a computer program in accordance with claim 4.

6. Method for quantitative assessment of radionuclide levels in the bloodstream of a subject following administration of said radionuclide to said subject, said method comprising:
acquiring an input radionuclide signal using a gamma scintillation counter with a probe placed over a portion of the subject's body;
feeding the input radionuclide signal to a processing unit;
phase sensitive processing, by said processing unit, of the input radionuclide signal, extracting from said input radionuclide signal an output signal representing the radionuclide concentration in the bloodstream, wherein the phase sensitive processing uses the phase and frequency of arterial vessel volume changes as a reference waveform.

7. Method according to claim 6, wherein the phase and frequency of the arterial vessel volume changes are derived from the signal of the gamma scintillation counter.

8. Method according to claim 6, wherein the phase and frequency of the arterial vessel volume changes are determined using near-infrared reflectance (NIR) measurement, using a NIR probe placed over a portion of the subject's body.

9. Method according to claim 6 any wherein the radionuclide is a ¹⁸F radionuclide.

10. Method according to claim 6 any wherein the radionuclide is administered through intravenous injection.

11. Method according to claim 6 any wherein said portion of the subject's body is selected from the upper arm, the elbow, the lower arm, the wrist, the hand, fingers, the neck, or the limbs.

12. Method of quantitative assessment of radionuclide uptake by a tissue in a subject using positron emission tomography (PET) or Single Photon Emission Computed Tomography (SPECT) imaging performed after administration of said radionuclide to said subject, wherein the time course of activity concentration (TCC) or arterial input function (AIF) is acquired using the method as defined in claim 1.

13. Method according to claim 12, wherein said tissue is selected from a tumor, tumorigenic tissue, brain tissue, vascular tissue and cardiac tissue.

14. Method according to claim 12, wherein the method is used to diagnose and/or stage cancers, monitor treatment of cancers; diagnose Alzheimer's disease; localize seizure focus; diagnose and/or study neuropsychiatric and neurologic illnesses; diagnose and/or study atherosclerosis and vascular disease; identify hibernating myocardium; study schizophrenia, substance abuse, mood disorders and/or other psychiatric conditions; study biodistribution in pre-clinical trial of new drugs; or study drug occupancy at a purported sites of action by competition studies.

15. System according to claim 2, wherein the gamma scintillation counter probe is adapted to be placed over a subject's finger.

16. Method according to claim 7, wherein the radionuclide is a ¹⁸F radionuclide.

17. Method according to claim 7, wherein the radionuclide is administered through intravenous injection.

18. Method according to claim 7 wherein said portion of the subject's body is selected from the upper arm, the elbow, the lower arm, the wrist, the hand, fingers, the neck, or the limbs.

19. Method according to claim 13, wherein the method is used to diagnose and/or stage cancers, monitor treatment of cancers; diagnose Alzheimer's disease; localize seizure focus; diagnose and/or study neuropsychiatric and neurologic illnesses; diagnose and/or study atherosclerosis and vascular disease; identify hibernating myocardium; study schizophrenia, substance abuse, mood disorders and/or other psychiatric conditions; study biodistribution in pre-clinical trial of new drugs; or study drug occupancy at a purported sites of action by competition studies.
